(19) United States
(12) Patent Application Publication
(10) Pub. No.: US 2024/0285154 A1
(43) Pub. Date: Aug. 29, 2024

(54) ENDOSCOPE AND METHOD FOR OPERATION

(71) Applicant: LASER ZENTRUM HANNOVER E.V., Hannover (DE)

(72) Inventors: Björn SPOIDA, Hannover (DE); Sonja JOHANNSMEIER, Hannover (DE); Rammo RIPKEN, Wunstorf (DE)

(21) Appl. No.: 18/442,706

(22) Filed: Feb. 15, 2024

(30) Foreign Application Priority Data
Feb. 23, 2023 (DE) 10 2023 104 405.2

(51) Int. Cl. A61B 1/018; A61B 1/00; A61B 1/015; A61B 1/05; A61B 1/06; A61B 1/07

(52) U.S. Cl. CPC A61B 1/018; A61B 1/00096; A61B 1/00135; A61B 1/015; A61B 1/05; A61B 1/063; A61B 1/07

(57) ABSTRACT

An endoscope with an outer cladding tube has a working channel with a front opening and a rear opening. In a longitudinal direction, which extends from the rear opening to the front opening, the endoscope includes a working tube with a front end and a rear end. The rear end can be inserted into the rear opening of the working channel and includes at least one light guide channel for guiding laser radiation, at least one flushing channel for guiding a flushing gas and/or a flushing liquid, and at least one suction channel.

### FIELD OF THE INVENTION

The invention relates to an endoscope with an outer cladding tube, in which a working channel with a front opening and a rear opening and a longitudinal direction, which extends from the rear opening to the front opening, runs.

### BACKGROUND

Such endoscopes have been known from the prior art for many years. They are also referred to as medical endoscopes and used, for example, for operations in which the wound inflicted on the patient should be as small as possible. This is known as minimally invasive medicine. Such endoscopes have a working channel through which the actual medical tool required for the respective operation is introduced into the patient's body through the cladding tube. There is often also a lighting channel in the cladding tube through which light from an often external light source can be guided through the cladding tube to the actual operation area. In this case, an observation channel is usually provided as well, through which the operator can see through the cladding tube and observe the actual operation area inside the patient's body, said operation area being illuminated by light that has been guided through the lighting channel.

However, endoscopes are also used in operations in which tight and narrow cavities have to be penetrated or work is to be performed inside such cavities. This is the case, for example, when a hip prosthesis is to be removed from the patient's bone. A hip prosthesis is an endoprosthesis that is arranged in the patient's body and replaces the patient's natural hip joint. They are therefore often referred to as total hip endoprostheses. Such a prosthesis comprises a prosthesis socket, which is anchored in the patient's femur; a round prosthesis head, which is positioned at the proximal end of the prosthesis socket, i.e. the end facing the torso; an inlay arranged thereon, which acts as a sliding component; and an implant of the acetabulum. The femur is a long bone with an inner cavity into which the prosthesis socket of the hip prosthesis is inserted. The cavity is milled beforehand to create sufficient space for the prosthesis socket. To achieve the best possible adhesion between the prosthesis socket used and the bone, many operations use a so-called bone cement between the bone and the prosthesis socket, which acts as a glue.

There are a number of medical indications, for example loosening of the prosthesis, that lead to a hip prosthesis or another total endoprosthesis having to be replaced. In this case, the prosthesis socket must be removed from the femur and replaced by another prosthesis socket. The problem with this process is the bone cement that remains in the bone, which also has to be removed before a new prosthesis socket can be deployed. This is done, for example, by means of a chisel or a tool that has a blade. The problem with this and other methods is that, when removing the respective part of the bone cement, it is not possible to observe the area and position being processed. As a result, it is not only the bone cement within that is removed, but also part of the bone, for example, which is disadvantageous regarding the stability of the bone and can also have a negative impact on a hip prosthesis used at a later date. This often occurs when larger fragments of cement are chipped off or deposited. In such mechanical processes, the mechanical load caused by the applied forces is also very high, which presents an additional risk regarding the stability of the remaining bones.

A wide array of devices are known from the prior art which can be used to remove the bone cement in a different way. In particular, attempts have been made to do this through laser ablation, where the cement is irradiated with laser radiation and thus removed. However, such methods have the disadvantage that the thermal load of surrounding tissue or bone material is very high, which can result in permanent damage. A device intended for this purpose can be found, for example, in WO 2011/109414 A2. For example, DE 3 212 180 discloses an endoscopic laser coagulation unit in which laser radiation is guided to the front end of the endoscope by means of a laser beam guide fiber, wherein the laser light does not exit along the longitudinal direction of the endoscope, but at an angle of 6° to this longitudinal direction. U.S. Pat. No. 6,620,154 B1 presents a similar device in which an optical element is arranged at the front end of the endoscope, with which the laser light guided through the endoscope exits at an angle of 90° to the longitudinal direction of the endoscope. The device described in this publication is intended for eye operations.

The disadvantage is that the mechanical removal of the remaining bone cement in the bone using a hammer and chisel is often imprecise and difficult to control. This leads to increased damage to the bone, which renders the implantation of a subsequent prosthesis more difficult. Conversely, removal using laser radiation is complex and time-consuming. A lengthy operation is disadvantageous for both the operating surgeon, as it is strenuous and said person is tired, and for the patient, as the necessary long anaesthesia increases the risks for the patient. Ultimately, a long operation is also a disadvantage from an economic perspective, given that the longer the operation takes, the more the costs increase. In addition, the vaporization of the bone cement results in poisonous or at least harmful gases, which have to be carefully removed from an operating theater. The invention is therefore based on the task of eliminating or at least reducing the disadvantages of the prior art.

### SUMMARY

The invention solves the addressed task by way of an endoscope which is characterized in that it comprises a working tube with a front end and a rear end, which can be inserted into the rear opening of the working channel and comprises at least one light guide channel for guiding laser radiation, at least one flushing channel for guiding a flushing gas and/or a flushing liquid, and at least one suction channel.

When the endoscope is in use, the front opening of the working channel is inside the patient, for example inside a femur. The rear opening, through which the working tube can be inserted into the endoscope, is also accessible to the operating surgeon during the operation. It is therefore possible to remove the working tube from the cladding tube of the endoscope during the operation or to insert it into the cladding tube. This means, for example, that a working tube can be replaced by another working tube. The working tube contains a light guide channel, through which laser radiation, which is preferably generated by a laser located outside of the endoscope, is guided through the working tube to the desired point, for example inside the femur. This laser radiation can be used to process, for example remove or vaporize, bone cement, for instance, or another substance with the laser. Any possible resulting products, such as gases, are likewise flushed out through the working tube. To this end, a flushing gas or flushing liquid is guided through the at least one flushing channel. The flushing gas and/or flushing liquid leave through the front end of the working tube and therefore also through the front opening of the working channel and flush the working space in front of it. The flushing gas and/or flushing liquid leave the working space through the at least one suction channel with the material that is to be removed by the flushing process.

In a preferred embodiment, the rear end of the working tube has a connection device by way of which the rear end of the light guide channel can be connected to a light conductor, such as a glass fiber, through which laser radiation from an external laser can be guided to the working tube. This connection device is preferably attached directly to the light guide channel, especially preferably at its rear end. The working tube preferably has at least one connection device by way of which the rear end of the flushing channel can be connected to a gas or liquid line. A flushing gas or flushing liquid is guided through this gas or liquid line from an external gas or liquid source to the flushing channel. This connection device is preferably attached directly to the flushing channel, especially preferably at its rear end. The working tube preferably has at least one connection device by way of which the rear end of the suction channel can be connected to a suction line. This suction line allows the suction channel to be connected to a suction source, such as a pump, by way of which the suction line can be subjected to a negative pressure, so that material located in front of the front end of the suction channel, i.e. in the working space, is extracted through the suction channel. Preferably, this connection device is located directly on the suction channel, particularly preferably at its rear end.

The light guide channel is preferably designed as an optical fiber, especially a glass fiber, and/or as a waveguide and/or a free beam. Particularly if an optical fiber is used, it is advantageous if it is surrounded by a stabilizing sheath, for example a metal tube.

In some embodiments of the invention, the light guide channel is closed towards the front end of the working tube by an optical component, in particular a lens or a prism. The light guide channel has a front end, which is arranged in the area of the front end of the working tube. The optical component may also be designed as a light-emitting surface of an optical fiber or as a penetration surface of an optical fiber, such as a glass fiber. The optical component closes the light guide channel towards the front end, so that no liquids and/or gases are able to penetrate the light guide channel, particularly when the endoscope is in use, for example during an operation. This is especially important and advantageous when the optical fiber is designed as a waveguide or a free beam. Closing the light guide channel as hermetically as possible makes it easier to clean the endoscope, or at least the working tube, after use and, if necessary, to sterilize it so that it can be used again. The optical component is preferably a collimator or a focussing component.

In a preferred embodiment, the optical component is designed in such a way that light that is directed through the light guide channel and passes through the optical component leaves the optical component along an exit direction, which is at an angle of at least 10°, preferably at least 30°, to the longitudinal direction of the working channel. Preferably, the endoscope has multiple working tubes, each of which has an optical component at the front end of the light guide channel and which create different exit directions. This means that, during an operation, a working tube can initially be used whose optical component creates a first exit direction at the front end of the light guide channel. At a later point in the operation, this first working tube can be removed from the rear opening of the working channel of the endoscope and replaced by a second working tube. This second working tube is then inserted into the rear opening of the working channel. The second working tube preferably has an optical component at the front end of the light guide channel, which creates a second exit direction for the laser light, said exit direction being different from the first exit direction.

In a preferred embodiment, the exit direction extends, preferably parallel, at an angle of less than 10° to the longitudinal direction of the working channel. This is particularly advantageous if an area is to be processed that is located in front of the front end of the working tube in the longitudinal direction. An embodiment of the invention includes a first working tube, the exit direction of which extends at an angle of less than 10°, preferably 0°, to the longitudinal direction of the working channel, and a second working tube, the exit direction of which extends at an angle of approximately 30° to said longitudinal direction of the working channel.

The optical component can be designed in different ways. Preferably, it contains a mirror and/or a prism and/or a grid, which are arranged in such a way that laser light that is directed through the light guide channel is deflected by said component. In a preferred embodiment, the optical component is designed to be movable, which can be achieved, for example, by way of a tilting and/or rotating mirror. In this preferred embodiment, the exit direction, along which the laser light directed through the light guide channel leaves the optical component, can be changed and adjusted.

In a preferred embodiment, in one section of the working tube the light guide channel does not extend parallel to the working tube, said section preferably being located immediately in front of the front end of the working tube. This is particularly advantageous if the light guide channel is designed as an optical fiber. The exit direction in which light leaves the optical fiber is largely determined by the direction along which the light guide channel extends in the last section through which the light is guided. The section is preferably several centimeters long, for example 2 cm, 3 cm or 5 cm. In a preferred embodiment, the light guide channel does not extend in a straight line along the section, but in a curve. In said section, it preferably has a constant radius of curvature. The longer the section, the greater the radius of curvature can be, wherein a greater radius of curvature is advantageous as the optical fiber is subjected to less mechanical stress and the scattering losses caused by laser radiation escaping from the cladding surface of the optical fiber are reduced.

A shape of the cross-sectional surface of the flushing channel and/or the suction channel preferably changes in said section, wherein the surface area of the cross-sectional surface is preferably constant. The flushing channel and/or the suction channel preferably extend parallel to the working tube. Particularly preferably, the flushing channel and/or the suction channel run parallel to the working tube across the entire length of the working tube. However, if the light guide channel in the aforementioned section is not parallel to the working tube, it may be necessary or at least advantageous if the shape of the cross-sectional surface of the flushing channel and/or the suction channel changes in order to make room for the respective light guide channel, which runs at different points in the cross-sectional surface of the working tube.

In a preferred embodiment, an observation device is provided in the outer cladding tube, preferably in the working tube, said device being configured to detect light entering the observation device from a predetermined solid angle range relative to the longitudinal direction of the working channel. Preferably, the observation device contains an observation channel, which is preferably designed as an optical fiber, for example a glass fiber. The light that is in the working space in front of the front opening of the working channel and that enters into the front end of the optical fiber in the respective solid angle range is guided through the observation channel by said optical fiber. For example, it reaches an optical sensor, such as a CCD chip or a camera, or is guided through an optical device, such as a microscope, so that the surgeon using the endoscope can see into the working space.

Preferably, the optical sensor is arranged at the front end of the outer cladding tube, preferably at the front end of the working tube, so that light directly from the working space can reach the optical sensor without being guided by an optical fiber. The signals generated by the optical sensor, which can be electronic signals, for example, are transmitted through a data cable to an electronic data processing device. This data cable can also be referred to as an observation channel. Communication between the optical sensor and the electronic data processing device can be achieved in a wired or wireless manner, for example via radio communication, WiFi or Bluetooth.

Particularly preferably, the exit direction along which light leaves the light guide channel lies in the predetermined solid angle range, the predetermined solid angle range preferably being symmetrical about the exit direction. The exit direction lying within the predetermined solid angle range ensures that the working area where the exiting laser light strikes an object or a tissue can be observed by way of the observation device. This allows the working area to be observed while the endoscope is in use.

The observation device preferably has a light detector, such as a CCD chip. Particularly preferably, said chip is arranged in such a way that light entering through the front opening strikes the light detector either directly or through an optical device focussed on said detector. The resulting electrical signals are guided through the outer cladding tube, preferably through the working tube, through an electrical line or signal line, which also constitutes part of the observation device. Preferably, the outer cladding tube, especially preferably the working tube, has a connection device with which the connection to an external data cable can be established, via which the signals and working data generated by the light detector are fed to a processing device, preferably an electronic data processing device.

It has been proven advantageous if there is a lighting device inside the endoscope, said device being configured to illuminate a working space, which is located in front of the front opening of the working channel, with electromagnetic radiation. The lighting device is designed, for example, in the form of an optical fiber, such as a glass fiber. It extends through the working channel or outside of the working channel through the cladding tube. There is preferably a connection device arranged at the end of the optical fiber facing the rear opening of the working channel through which light can be introduced into the optical fiber. In this case, light refers to any electromagnetic radiation that can be used for illumination. Alternatively or additionally, the lighting device has a light source, such as an LED, which is arranged in the outer cladding tube, for example in the working channel or outside of the working channel, and is connected to a power supply by way of an electrical line that runs through the working channel or outside of the working channel, but inside the cladding tube. As a result, the light required for illumination can be generated in the area of the front opening of the working channel, thereby preferably illuminating the entire working space.

Preferably, the working channel and/or the flushing channel and/or the suction channel are designed in the form of a tube, preferably a metallic tube. The tube preferably has a circular cross section at least in sections, but especially preferably across its entire length. However, the cross section may also be oval, ellipsoid or free-form. Preferably, a diameter, a radius of curvature and/or a geometric form of the cross section changes depending on the position along the longitudinal direction of the working channel. The surface area of the cross-sectional surface preferably remains constant over the entire length of the respective channel.

In a preferred embodiment, the working tube has multiple flushing channels, preferably at least 2, at least 3 or at least 4. Particularly preferably, each of said multiple flushing channels is connected to a source of flushing gas or flushing liquid. Advantageously, each individual flushing channel has a valve through which the through-flow of the flushing gas and/or the flushing liquid can be allowed and prevented. Particularly preferably, the valves can be controlled independently of each other, so that the individual flushing channels can be opened and closed independently of each other. It is therefore possible to change the direction and/or speed of flow of the flushing medium within the working space in front of the front opening of the working channel. For example, if it transpires that the flushing medium used moves, for example, a gas resulting from laser radiation in such a way that sufficient light no longer enters the observation device, the number or selection of the flushing channels through which the flushing medium, i.e. the flushing gas and/or the flushing liquid, is guided into the working space can be changed by closing and/or opening individual flushing channels. The flow circumstances inside the working space change as a result.

Advantageously, the front end of the working tube is closed by an end cap that all channels are guided through. This prevents, for example, liquids or gases from entering into the working channel through the front opening. It facilitates cleaning and disinfection of the endoscope, so that it can be reused more quickly and easily. The end cap is also configured to align the respective ends of the channels with each other and to fix their position relative to each other. Particularly preferably, the end cap protrudes in the longitudinal direction of the working channel beyond the optical component at the front end of the light guide channel. This protects the often sensitive optical component from mechanical damage.

The working tube preferably has a locking device, with which is can preferably be locked in the working channel in multiple predetermined orientations, preferably in a single orientation. The lockability means that the endoscope can be moved as a whole, in particular with the working tube located inside it. The endoscope preferably has a handle by way of which the operator can touch and handle the endoscope. If the handle, which is preferably rigidly connected to the outer cladding tube, is moved, said cladding tube will also move. As a result, the working tube and the channels and devices extending within it are also moved, provided that the working tube with the working channel is locked. If the working channel can only be locked in a single orientation relative to the working channel, incorrect adjustments and orientations can be reliably avoided.

The invention also solves the addressed task by way of working tube for an endoscope described here.

The invention also solves the addressed task by way of a method for operating an endoscope described here, the method comprising laser radiation being guided through the light guide channel and a flushing medium, preferably a flushing gas and/or flushing liquid, being guided through the flushing channel and extracted through the suction channel. In a preferred embodiment of the method, the laser radiation has a wavelength of 2013+100 nm and/or an output of 5 W to 150 W and/or the laser radiation is pulsed laser radiation with a pulse length of 200 μs to 1 μs. In other embodiments of the invention, a laser with a wavelength of 3 μm or a CO₂ laser with a wavelength of 10.6 μm is used.

### DESCRIPTION OF THE DRAWINGS

In the following, a number of embodiment examples of the invention will be explained in more detail with the aid of the accompanying drawings. They show

FIGS. 1 to 4 schematic sectional views through various embodiments of a working tube,

FIG. 5 the schematic representation of an endoscope and

FIG. 6 an enlarged section from FIG. 5.

### DETAILED DESCRIPTION

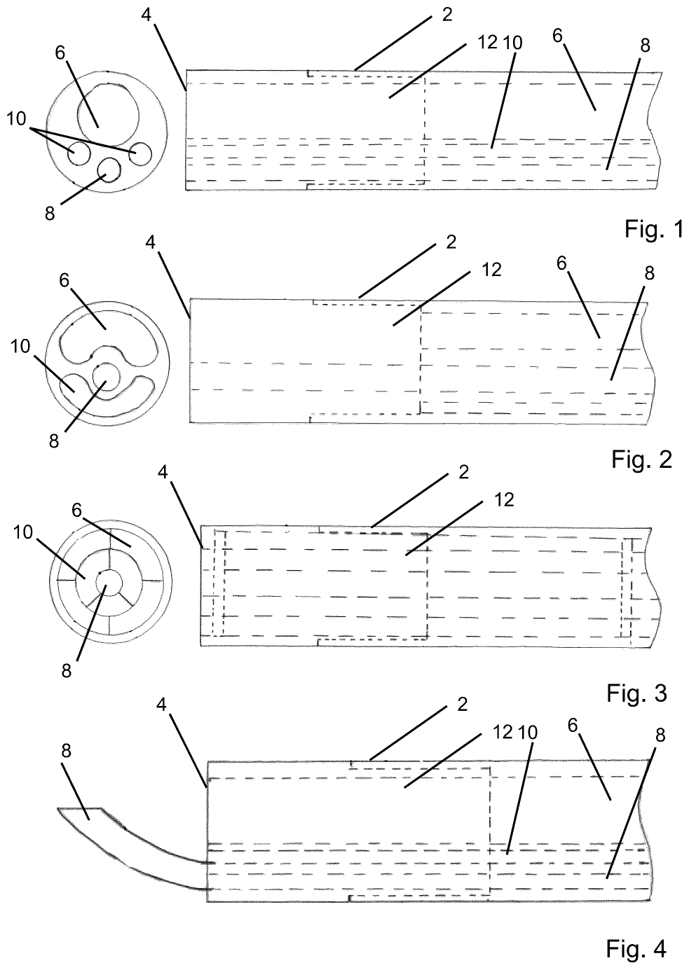

FIGS. 1 to 3 schematically depict a sectional view through a working tube 2 as can be used in endoscopes according to embodiments of the present invention. In addition, a frontal view of a front end 4 of the working tube 2 is shown in the left section of each of the FIGS. 1 to 3. In the sectional view of the working tube 2, different channels located inside the working tube 2 are illustrated with dashed lines.

In FIG. 1, the working tube 2 has a suction channel 6, a light guide channel 8 as well as 2 flushing channels 10. In the left-hand area of FIG. 1, one can see that the suction channel 6 has the largest cross section of all the channels. The two flushing channels 10 are arranged to the right and left of the light guide channel 8. A flushing gas or flushing liquid is guided through said channels into the working space, which is located in front of the front end 4 of the working tube 2. The flushing gas or flushing liquid therefore leaves the flushing channels 10 and is removed again from the working space through the suction channel 6. The arrangement of the flushing channels 10 in relation to the suction channel 6 ensures that laser light leaving the light guide channel 8 that is not used to process the material in the working space is not affected by the flushing gas or flushing liquid. The front end 4 of the working tube 2 is closed by an end cap 12, which is likewise illustrated by a dashed line. The suction channel 6, the light guide channel 8 and the flushing channels 10 extend through the end cap 12 and are preferably stabilized by the end cap 12.

FIG. 2 shows a different embodiment. In this case, the front end 4 of the working tube 2 is also closed by the end cap 12 through which the suction channel 6, the light guide channel 8 and the flushing channel 10 are guided. However, one can see in the left-hand section of FIG. 2 that the arrangement of the various channels is different compared to the depiction in FIG. 1. The light guide channel 8 is arranged almost centrally and is at least partially surrounded by the suction channel 6 and the flushing channel 10. This alters the flow properties so that flushing gas or flushing liquid exiting through the flushing channel 10, which leaves the working space again through the suction channel 6, affects the light exiting the light guide channel 8 as little as possible.

FIG. 3 shows a further embodiment of a front end 4 of a working tube 2 closed by the end cap 12. In the left-hand section of FIG. 3, one can see that the different types of channels are arranged concentrically. The light guide channel 8 is positioned centrally. Said channel is surrounded by a flushing channel 10, which is constructed of three segments that completely surround the light guide channel 8 in the embodiment example shown. The lines shown between adjacent segments merely correspond to stabilizing walls that lend stability to the end cap 12. There are four suction channels 6 located radially outwards, which form the outer channel ring.

FIG. 4 depicts a representation similar to the embodiment in FIG. 1. The main difference, however, is that the light guide channel 8 protrudes beyond the front end 4 of the working tube 2, where it is curved. This renders it possible for light exiting the light guide channel 8 to exit not along the longitudinal direction of the working tube, i.e. from right to left in FIGS. 1 to 4, but at a different angle.

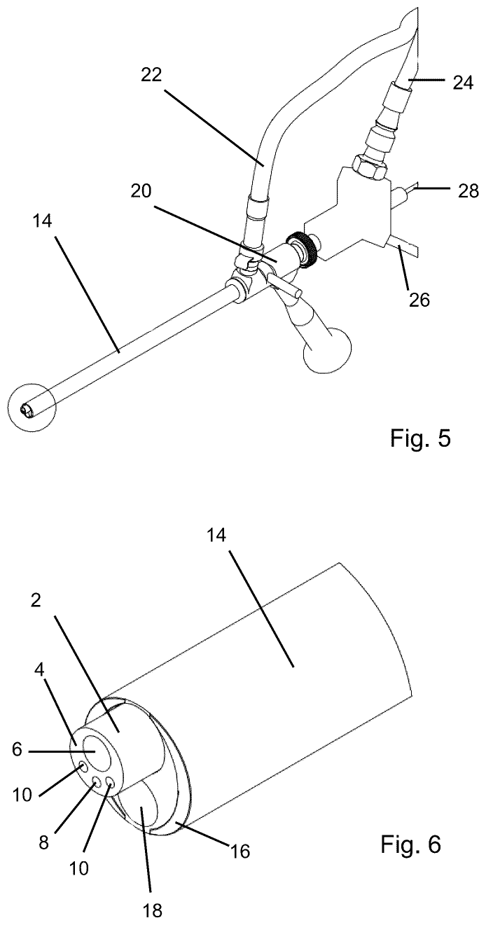

FIG. 5 schematically depicts an endoscope according to an embodiment example of the present invention. FIG. 6 shows an enlarged section from FIG. 5, namely the front end of the endoscope. The endoscope has an outer cladding tube 14, into which the working tube 2 is inserted. FIG. 6 shows the front end. One can recognize the working tube 2, at the front end of which the light guide channel 2, the suction channel 6 and the two flushing channels 10 are visible. In addition, a lighting channel 16 and an observation channel 18 are arranged in the outer cladding tube 14. Illumination light is guided through the lighting channel 16 into the working space in front of the front end 4, so that the operator can observe the processing space when the endoscope is in use. The illumination light illuminates the working space and reflected illumination light re-enters the outer cladding tube 14 through the observation channel 18.

FIG. 5 illustrates that a coupling element 20 is located at the rear end of the outer cladding tube 14, the various channels located in the outer cladding tube 14 and in particular in the working tube 2 being connected to different supply lines in said coupling element. To this end, the endoscope has a light line 22, which is preferably connected to a light source at its end that is not depicted. Said light source emits illumination light, which is guided through the light line 22 to the coupling element 20 and from there into the lighting channel 16. A suction connection 24 is preferably connected to a negative pressure source, such as a pump. Said source generates a negative pressure and therefore a suction effect, which is transferred to the suction channel 6 via the coupling element 20. Material that is extracted through the suction channel 6 is fed to the suction connection 24 via the coupling element 20. Flushing gas or flushing liquid is fed via a flushing connection 26 to the coupling element 20 and via the latter to the flushing channel 10. The laser light required for the laser processing, which is fed to the working space via the light guide channel 8, enters the coupling element 20 via a laser connection 28 and from there it enters the light guide channel 8.

### REFERENCE LIST

2 working tube
4 front end
6 suction channel
8 light guide channel
10 flushing channel
12 end cap
14 outer cladding tube
16 lighting channel
18 observation channel
20 coupling element
22 light line
24 suction connection
26 flushing connection
28 laser connection

1. An endoscope, comprising:
an outer cladding tube;
a working channel which runs in the outer cladding tube and extends in a longitudinal direction from a rear opening to a front opening; and
a working tube with a front end and a rear end which is insertable into the rear opening of the working channel, wherein the working tube comprises
at least one light guide channel for guiding laser radiation,
at least one flushing channel for guiding a flushing gas and/or a flushing liquid, and
at least one suction channel.

2. The endoscope according the claim 1, wherein the light guide channel is designed as an optical fiber and/or as a waveguide and/or a free beam.

3. The endoscope according to claim 1 wherein the light guide channel is closed towards the front end of the working tube by an optical component.

4. The endoscope of claim 3 wherein the optical component is a lens or a prism.

5. The endoscope according to claim 3 wherein the optical component is designed such that light that is guided through the light guide channel and passes through the optical component exits the optical component along an exit direction which is at an angle of at least 10° to the longitudinal direction of the working channel.

6. The endoscope according to claim 5 wherein the angle is at least 30° to the longitudinal direction of the working channel.

7. The endoscope according claim 1 wherein in one section of the working tube the light guide channel does not extend parallel to the working tube.

8. The endoscope according to claim 7 wherein the one section is located immediately in front of the front end of the working tube.

9. The endoscope according to claim 7, wherein a shape of a cross-sectional surface of the flushing channel and/or the suction channel changes in the one section.

10. The endoscope of claim 9 wherein a surface area of the cross-sectional surface is constant.

11. The endoscope according to claim 1 further comprising an observation device provided in at least one of the outer cladding tube and the working tube, wherein the observation device is configured to detect light entering the observation device from a predetermined solid angle range relative to the longitudinal direction of the working channel.

12. The endoscope according to claim 11 wherein an exit direction lies within the predetermined solid angle range.

13. The endoscope according to claim 12 wherein the predetermined solid angle range is symmetrical about the exit direction.

14. The endoscope according to claim 11 wherein the observation device comprises a light detector.

15. The endoscope according to claim 1 wherein the working channel and/or the flushing channel and/or the suction channel are designed a tubes.

16. The endoscope according to claim 1 wherein the working tube comprises multiple flushing channels.

17. The endoscope according to claim 1 wherein the front end of the working tube is closed by an end cap, and wherein the at least one guide channel, the at least one flushing channel, and the at least one suction channel are guided through the end cap.

18. The endoscope according to claim 17 further comprising an optical component located toward the front end of the working tube, and wherein the end cap protrudes in the longitudinal direction of the working channel beyond the optical component.

19. The endoscope according to claim 1 further comprising locking device associated with the working tube, wherein the locking device is configured to lock the working tube to the working channel in one or more predetermined orientations.

20. A working tube for an endoscope according to claim 1.

21. A method for operating an endoscope according to claim 1, comprising:
guiding laser radiation through the light guide channel;
guiding a flushing gas and/or flushing liquid through the flushing channel; and
extracting material or fluid through the suction channel.

22. The method according to claim 21, wherein the laser radiation has a wavelength of 2013+100 nm and/or an output of 5 W to 150 W and/or is pulsed laser radiation with a pulse length of 200 μs to 1 μs.
